(19) United States
(12) Patent Application Publication
(10) Pub. No.: US 2024/0153631 A1
(43) Pub. Date: May 9, 2024

(54) ELECTRONIC DEVICE AND METHOD FOR PROVIDING RECOMMENDED DIAGNOSIS

(71) Applicant: AESOP Technology Inc., San Francisco, CA (US)

(72) Inventor: An Jim Long, Taipei City (TW)

(73) Assignee: AESOP Technology Inc., San Francisco, CA (US)

(21) Appl. No.: 18/279,875

(22) PCT Filed: Aug. 19, 2021
(86) PCT No.: PCT/US2021/046587

§ 371 Date: Sep. 1, 2023

Related U.S. Application Data
(60) Provisional application No. 63/196,186, filed on Jun. 2, 2021.

(51) Int. Cl. G16H 50/20; G16H 10/60; G16H 15/00; G16H 50/30; G16H 70/60

(52) U.S. Cl. CPC G16H 50/20; G16H 10/60; G16H 15/00; G16H 50/30; G16H 70/60

(57) ABSTRACT

An electronic device and a method for providing a recommended diagnosis are provided. The method includes the following. A first similarity between a first diagnosis and a first medical parameter is obtained. A medical record of a patient is obtained. In response to the medical record including the first medical parameter, a recommended diagnosis list corresponding to the medical record is generated based on the first similarity, and the recommended diagnosis list includes the first diagnosis corresponding to the first similarity. The recommended diagnosis list is output.

### CROSS-REFERENCE TO RELATED APPLICATION

This application claims the priority benefit of U.S. provisional application Ser. No. 63/196,186, filed on Jun. 2, 2021. The entirety of the above-mentioned patent application is hereby incorporated by reference herein and made a part of this specification.

### BACKGROUND

### Technical Field

The disclosure relates to a clinical medical technology, and in particular to an electronic device and a method for providing a recommended diagnosis.

### Description of Related Art

In recent years, artificial neural networks have become one of the mainstream technologies in the fields of artificial intelligence and big data. However, in the medical field where there are tens of thousands of features, artificial neural network models are often unable to generate accurate prediction results due to insufficient training samples. In the case of hospitalization, often, there are only millions of samples that may be collected clinically while there are tens of thousands of features. Therefore, an artificial neural network trained based on these samples often fails to achieve good performance.

### SUMMARY

The disclosure provides an electronic device and a method, which provide a recommended diagnosis for a user's reference.

An electronic device for providing a recommended diagnosis of the disclosure includes a processor, a storage medium, and a transceiver. The storage medium stores a first similarity between a first diagnosis and a first medical parameter. The processor is coupled to the storage medium and the transceiver, and the processor is configured to execute: receiving a medical record of a patient through the transceiver; in response to the medical record including the first medical parameter, generating a recommended diagnosis list corresponding to the medical record based on the first similarity, wherein the recommended diagnosis list includes the first diagnosis corresponding to the first similarity; and outputting the recommended diagnosis list through the transceiver.

In an embodiment of the disclosure, the storage medium mentioned above further stores a second similarity between the first diagnosis and a second medical parameter, and the processor is configured to execute: in response to the medical record including the second medical parameter, generating the recommended diagnosis list based on the second similarity.

In an embodiment of the disclosure, the above-mentioned processor is further configured to execute: calculating a first similarity coefficient based on the first similarity and the second similarity, and selecting the first diagnosis based on the first similarity coefficient to generate the recommended diagnosis list.

In an embodiment of the disclosure, the above-mentioned processor adds the first similarity and the second similarity to generate the first similarity coefficient.

In an embodiment of the disclosure, the above-mentioned processor is further configured to execute: in response to the first similarity coefficient corresponding to the first diagnosis being greater than a second similarity coefficient corresponding to a second diagnosis, selecting the first diagnosis from the first diagnosis and the second diagnosis as a selected diagnosis corresponding to the first medical parameter; and generating the recommended diagnosis list based on the selected diagnosis.

In an embodiment of the disclosure, the above-mentioned medical record includes multiple medical parameters, and the processor is further configured to execute: calculating a number of selected diagnosis based on a weight corresponding to the first medical parameter; selecting the first medical parameter from the plurality of medical parameters based on the number of selected diagnosis to obtain a selected diagnosis corresponding to the first medical parameter; and generating the recommended diagnosis list based on the selected diagnosis.

In an embodiment of the disclosure, the above-mentioned processor determines the weight corresponding to the first medical parameter based on a department corresponding to the medical record.

In an embodiment of the disclosure, the storage medium mentioned above further stores a list corresponding to the first diagnosis, and the processor is configured to execute: obtaining an additional diagnosis corresponding to the first diagnosis based on the list; and adding the additional diagnosis to the recommended diagnosis list.

In an embodiment of the disclosure, the storage medium mentioned above further stores a blacklist corresponding to the first diagnosis, and the processor is further configured to execute: removing a recommended diagnosis in the recommended diagnosis list based on the blacklist.

In an embodiment of the disclosure, the above-mentioned processor accesses an external server through the transceiver to add updated data to the medical record.

In an embodiment of the disclosure, the above-mentioned processor outputs a report including the updated data through the transceiver based on a default text format.

In an embodiment of the disclosure, the above-mentioned first medical parameter corresponds to one of the following variables: patient features, inspections, medications, and treatment/surgery.

In an embodiment of the disclosure, the aforementioned storage medium further stores insurance data, and the processor calculates a cost of the first diagnosis based on the insurance data, and outputs a report including the cost through the transceiver.

A method for providing a recommended diagnosis of the disclosure includes the following. A first similarity between a first diagnosis and a first medical parameter is obtained. A medical record of a patient is obtained. In response to the medical record including the first medical parameter, a recommended diagnosis list corresponding to the medical record is generated based on the first similarity, and the recommended diagnosis list includes the first diagnosis corresponding to the first similarity. The recommended diagnosis list is output.

Based on the above, the electronic device of the disclosure determines the type of diagnosis that has a high similarity (a high similarity coefficient) with the patient based on the similarity between medical parameters and diagnosis, so as to provide a user with a recommended diagnosis list for the patient. The user selects an appropriate diagnosis to execute for the patient based on the recommended diagnosis list. The electronic device of the disclosure accurately determines the type of diagnosis associated with the patient without using a neural network. Therefore, the disclosure overcomes the problem of not being able to provide an appropriate diagnosis for patients due to insufficient samples and excessive variables of clinical data.

### BRIEF DESCRIPTION OF THE DRAWINGS

FIG. 1 illustrates a schematic diagram of an electronic device for providing a recommended diagnosis based on an embodiment of the disclosure.

FIG. 2 illustrates a flow chart of a method for providing a recommended diagnosis based on an embodiment of the disclosure.

### DESCRIPTION OF THE EMBODIMENTS

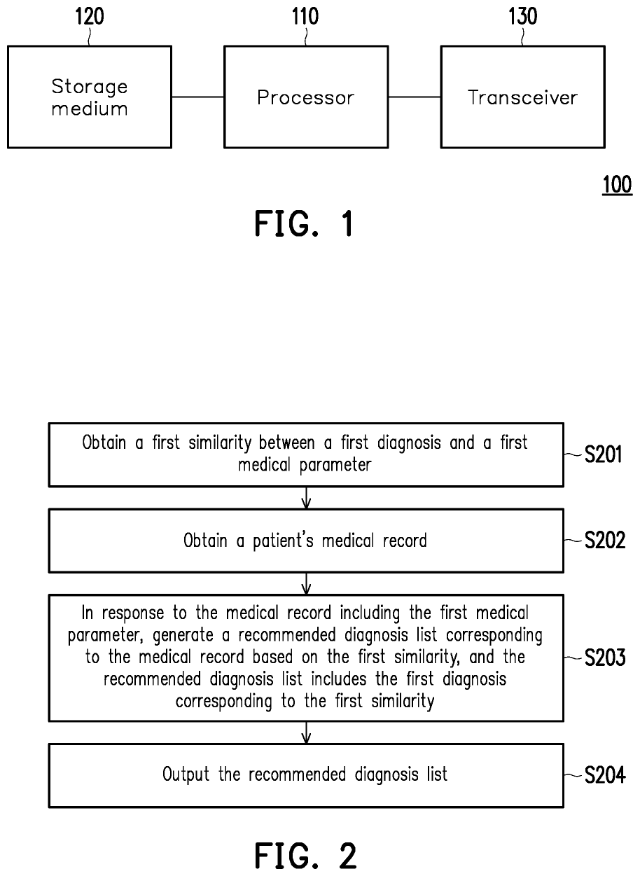

FIG. 1 illustrates a schematic diagram of an electronic device for providing a recommended diagnosis 100 based on an embodiment of the disclosure. The electronic device 100 may include a processor 110, a storage medium 120, and a transceiver 130.

The processor 110 is, for example, a central processing unit (CPU), or other programmable general-purpose or special-purpose elements including a micro control unit (MCU), a microprocessor, a digital signal processor (DSP), a programmable controller, an application specific integrated circuit (ASIC), a graphics processing unit (GPU), an image signal processor (ISP), an image processing unit (IPU), an arithmetic logic unit (ALU), a complex programmable logic device (CPLD), a field programmable gate array (FPGA) or other like elements or a combination of the above elements. The processor 110 may be coupled to the storage medium 120 and the transceiver 130, and the processor 110 accesses and executes a plurality of modules and various applications stored in the storage medium 120.

The storage medium 120 is, for example, any type of fixed or removable element including a random access memory (RAM), a read-only memory (ROM), a flash memory, a hard disk drive (HDD), a solid state drive (SSD) or other like elements or a combination of the above elements. The storage medium 120 is used to store a plurality of modules or various applications that may be executed by the processor 110.

The transceiver 130 transmits and receives signals in a wireless or wired manner. The transceiver 130 may further execute operations such as low noise amplification, impedance matching, frequency mixing, up or down frequency conversion, filtering, amplification, and the like.

The storage medium 120 may store a plurality of pieces of clinical data including similarities between diagnosis and medical parameters. The clinical data are obtained by the processor 110 through the transceiver 130 accessing an external big data database, for example. The medical parameters may include variables such as patient features, inspections, medications, or treatment/surgery, but the disclosure is not limited thereto.

The patient features may include variables such as a patient's department, race (White, African, Asian, Latino, etc.), socioeconomic level, height, weight, age, and gender. The inspections may include variables such as text, numbers, images, or signal waveforms. The medications may include variables such as the medications taken by the patient. The variables included in the medications may be given different weights based on the time of taking and the dosage. The treatment/surgery may include a treatment or surgery that the patient has undergone, and may include information such as a medical equipment used to perform the treatment or surgery.

Taking Table 1 as an example, Table 1 records a plurality of pieces of clinical data including the similarities between diagnosis and treatments. The similarities between diagnosis and treatments respectively are the similarity “2” between “Treatment 1” and “Diagnosis A”, the similarity “3” between “Treatment 1” and “Diagnosis B”, . . . , the similarity “10” between “Treatment 2” and “Diagnosis A”, and the similarity “3” between “Treatment 3” and “Diagnosis B”.

TABLE 1

| Treatment | Diagnosis | Similarity |
|---|---|---|
| Treatment 1 | Diagnosis A | 2 |
| Treatment 1 | Diagnosis B | 3 |
| . . . | . . . | . . . |
| Treatment 2 | Diagnosis A | 10 |
| treatment 3 | Diagnosis B | 3 |

It is worth noting that the processor 110 may encode medical parameters or recognize encoded medical parameters based on the following coding system: International Code of Disease-Procedure Coding System (ICD-PCS), Current Procedural Terminology (CPT), Healthcare Common Procedure Coding System (HCPCS), Code on Dental Procedures and Nomenclature (CDT), Health Insurance Prospective Payment System (HIPPS), RXNORM, National Drug Code (NDC), Anatomical Therapeutic Chemical (ATC) or Taiwan's National Health Insurance (NHI) Code, and the disclosure is not limited thereto.

The processor 110 may receive a patient's medical record through the transceiver 130. The medical record may include data that the hospital has registered for the patient. In an embodiment, the processor 110 may further access an external server through the transceiver 130 to add updated data to the medical record. For example, the processor 110 may add unregistered data to the medical record of the hospital so that the medical record is more complete. In an embodiment, the processor 110 may output a report (for example, a recommended diagnosis list) including the updated data through the transceiver 130 based on a default text format. For example, the processor 110 may output the medical record to display the medical record through an external display, and may display the updated data in the medical record in bold, red or special flags to remind a user that these updated data are newly added data that are not originally registered in the medical record.

In response to the medical record including a medical parameter, the processor 110 may generate a recommended diagnosis list corresponding to the medical record based on a similarity corresponding to the medical parameter, and the recommended diagnosis list may include a diagnosis corresponding to the similarity. The user may select one or more diagnosis from the recommended diagnosis list. Based on the diagnosis selected by the user, the hospital may perform a health check for the patient or make an insurance declaration for the patient.

Table 2 records the medical parameters included in the patient's medical record, which are respectively “Treatment 1” and “Treatment 2”. Taking Table 1 and Table 2 as examples, in response to the medical record including the medical parameter “Treatment 1”, the processor 110 may generate a recommended diagnosis list corresponding to the medical record based on the similarity “2” corresponding to “Treatment 1”, and the recommended diagnosis list may include “Diagnosis A” corresponding to the similarity “2”.

TABLE 2

Treatment 1
Treatment 2

Specifically, in response to the medical record including “Treatment 1” and “Treatment 2”, the processor 110 may calculate a similarity coefficient based on the similarities corresponding to “Treatment 1” and “Treatment 2” (that is, the similarity “2” corresponding to “Diagnosis A”, the similarity “3” corresponding to “Diagnosis B” and the similarity “10” corresponding to “Diagnosis A”). In other words, the processor 110 may find one or more diagnosis associated with the medical parameter recorded in the medical record from the storage medium 120, and calculate the similarity coefficient based on the diagnosis. The above-mentioned similarity is, for example, a conviction coefficient, but the disclosure is not limited thereto.

The processor 110 may calculate the similarity coefficient corresponding to “Diagnosis A” based on the similarity “2” and the similarity “10” corresponding to “Diagnosis A”. For example, the processor 110 may add up all the similarities (that is, the similarity “2” and the similarity “10”) corresponding to “Diagnosis A” to generate a similarity coefficient equal to “12”. On the other hand, the processor 110 may calculate the similarity coefficient corresponding to “Diagnosis B” based on the similarity “3” corresponding to “Diagnosis B”. For example, the processor 110 may add up all the similarities (that is, the similarity “3”) corresponding to “Diagnosis B” to generate a similarity coefficient equal to “3”.

The processor 110 may select a selected diagnosis from “Diagnosis A” and “Diagnosis B” based on the similarity coefficient “12” corresponding to “Diagnosis A” and the similarity coefficient “3” corresponding to “Diagnosis B”, and add the selected diagnosis to a list to generate the recommended diagnosis list. For example, in response to the similarity coefficient “12” corresponding to “Diagnosis A” being greater than the similarity coefficient “3” corresponding to “Diagnosis B”, the processor 110 may select “Diagnosis A” from “Diagnosis A” and “Diagnosis B” as the selected diagnosis.

The recommended diagnosis list may include a plurality of recommended diagnosis. Assuming that the medical record is associated with a plurality of diagnosis, in order to generate a plurality of recommended diagnosis, the processor 110 may generate a plurality of selected diagnosis based on a plurality of similarity coefficients respectively corresponding to a plurality of diagnosis. For example, assuming that the medical record is associated with “Diagnosis A”, “Diagnosis B” and “Diagnosis C”, and the similarity coefficient of “Diagnosis A” is greater than the similarity coefficient of “Diagnosis B”, and the similarity coefficient of “Diagnosis B” is greater than the similarity coefficient of “Diagnosis C”, if the processor 110 is to select two selected diagnosis from “Diagnosis A”, “Diagnosis B” and “Diagnosis C”, the processor 110 may, based on the similarity coefficients, sequentially select “Diagnosis A” with the largest similarity coefficient and “Diagnosis B” with the second largest similarity coefficient as the selected diagnosis.

When the medical record includes a plurality of medical parameters, among the medical parameters, the processor 110 may determine a medical parameter on which to be based to generate a plurality of recommended diagnosis based on the weight of various types of medical parameters, and the weight may be determined based on the department of the medical record. The processor 110 may calculate the number of selected diagnosis for a type of medical parameter based on the weight corresponding to the type of the medical parameter. For example, assuming that by default the recommended diagnosis list provides 10 recommended diagnosis, and the medical records of surgical patients include medical parameters including a plurality of treatments and a plurality of inspections, etc. In other words, the number of diagnosis by default in the recommended diagnosis list is “10”. For a department of surgery, the importance of treatment is higher than the importance of inspection, so the weight of treatment is set to “0.7”, and the weight of inspection is set to “0.3”. In this way, the processor 110 may multiply the default diagnosis number “10” by the weight of treatment “0.7” to generate a selected diagnosis number “7” corresponding to the treatments, and may multiply the default diagnosis number “10” by the weight of inspection “0.3” to generate a selected diagnosis number “3” corresponding to the inspections. In other words, the recommended diagnosis list includes 7 recommended diagnosis generated based on the treatments and 3 recommended diagnosis generated based on the inspections.

In an embodiment, the storage medium 120 may store a list corresponding to a recommended diagnosis, and the list may record one or more diagnosis corresponding to the recommended diagnosis. After generating the recommended diagnosis, the processor 110 may obtain an additional diagnosis corresponding to the recommended diagnosis based on the list of the recommended diagnosis, and add the additional diagnosis to the recommended diagnosis list. In other words, if a recommended diagnosis is a principal diagnosis, the processor 110 may add one or more additional diagnosis corresponding to the principal diagnosis to the recommended diagnosis list for the user's reference. For example, based on the surgery and treatment during a patient's hospitalization, the patient is known to have “diabetes” that is often accompanied by complications such as “foot ulcers” and “cellulitis”, so the storage medium 120 may store a list of diagnosis corresponding to “diabetes”, and the list may include the diagnosis of “foot ulcers” and the diagnosis of “cellulitis”. After the processor 110 determines that the diagnosis is recommended to be “diabetes”, the processor 110 may select additional diagnosis such as the diagnosis of “foot ulcers” and the diagnosis of “cellulitis” based on the list of diagnosis of “diabetes”. As another example, if a patient's prostate-specific antigen test result is positive, that is, the patient has a “prostate disease”, the processor 110 may select additional diagnosis such as “prostate enlargement with lower urinary tract symptoms” or “prostate inflammation” based on a list of diagnosis of “prostate disease”, and add these additional diagnosis to the recommended diagnosis list.

In an embodiment, the storage medium 120 may store a blacklist corresponding to a recommended diagnosis, and the blacklist may record one or more diagnosis corresponding to the recommended diagnosis. After generating the recommended diagnosis list, the processor 110 may remove the diagnosis in the blacklist from the recommended diagnosis list based on the blacklist of the recommended diagnosis. For example, since “hypertension” and “hypotension” do not occur to the same patient at the same time, the storage medium 120 may store a blacklist corresponding to the diagnosis of “hypertension”, and the blacklist may include the diagnosis of “hypotension”. After the recommended diagnosis list is generated, if the recommended diagnosis list includes both the diagnosis of “hypertension” and the diagnosis of “hypotension”, the processor 110 may remove the diagnosis of “hypotension” from the recommended diagnosis list based on the blacklist of the diagnosis of “hypertension”.

In an embodiment, the storage medium 120 may store information used for billing corresponding to the recommended diagnosis, such as insurance data, subsidy policy data, medical insurance rules adopted by a region, or a payment system for diagnosis related group (DRG). After obtaining the recommended diagnosis, the processor 110 may calculate the cost of the recommended diagnosis based on the information used for billing, and output a report (for example: a recommended diagnosis list) including the cost or the billing code for the user's reference.

A male patient over 60 years old suffered from long-term diabetes and thus had his left lower limb amputated. When the patient was admitted to the hospital, the medical record of the hospital recorded that the patient had undergone surgery such as skin resection of the left lower limb and skin resection of the left foot, and had taken or injected medications such as anesthetics and analgesics. In addition, the electronic device 100 obtains information such as medical materials used by the patient through accessing an external server, and may determine that the patient has also received medical treatments such as angioplasty and vascular stent placement based on the relationship between the medical materials and the medical treatments. In this case, the recommended diagnosis list of the patient may be as shown in Table 3.

TABLE 3

| Medications | Treatment/surgery |
|---|---|
| Anesthetics | Left lower limb skin resection |
| Analgesics | Left foot skin resection |
| | *Angioplasty |
| | *Vascular stent placement |
| Recommended diagnosis (principal diagnosis) | Recommended diagnosis (additional diagnosis) |
| Atherosclerosis of the left foot ($245) | Chronic and complete occlusion of arteries in |
| Diabetes with foot ulcers ($130) | extremities ($200) |
| Cellulitis ($80) | Acute Respiratory Distress Syndrome ($150) |
| Necrotizing Fasciitis ($60) | Homocystinuria ($100) |
| Skin abnormalities ($40) | Sepsis ($50) |
| | Diabetes with peripheral vascular disease ($30) |

Table 4 is a practical example of clinical data, and Table 5 is a practical example of a medical record. The processor 110 may calculate the similarity coefficient corresponding to tuberculosis to be “406.92” based on the similarities “187.56”, “8.82” and “210.54” corresponding to “tuberculosis”, may calculate the similarity coefficient corresponding to “pneumonia” to be “18.27” based on the similarities “16.84” and “1.43” corresponding to “pneumonia”, and may calculate the similarity coefficient corresponding to “chronic obstructive pneumonia” to be “1.51” based on the similarity “1.51” corresponding to “chronic obstructive pneumonia”. The processor 110 may select “tuberculosis” from “tuberculosis”, “pneumonia” and “chronic obstructive pneumonia” as a recommended diagnosis based on “tuberculosis” having the largest similarity coefficient.

TABLE 4

| Medications | Diagnosis | Similarity |
|---|---|---|
| Isoniazid | tuberculosis | 187.56 |
| Isoniazid | pneumonia | 16.84 |
| Metformin | chronic obstructive | 1.51 |
| | pneumonia | |
| Metformin | pneumonia | 1.43 |
| Metformin | tuberculosis | 8.82 |
| Streptomycin | tuberculosis | 210.54 |

TABLE 5

Isoniazid
Metformin
Streptomycin

FIG. 2 illustrates a flow chart of a method for providing a recommended diagnosis based on an embodiment of the disclosure, and the method may be implemented by the electronic device 100 shown in FIG. 1. In step S201, a first similarity between a first diagnosis and a first medical parameter is obtained. In step S202, a patient's medical record is obtained. In step S203, in response to the medical record including the first medical parameter, a recommended diagnosis list corresponding to the medical record is generated based on the first similarity, and the recommended diagnosis list includes the first diagnosis corresponding to the first similarity. In step S204, the recommended diagnosis list is output.

In summary, the electronic device of the disclosure may provide the user with a recommended diagnosis based on the similarity between objective medical parameters including patient features, inspections, medications, and treatment/surgery, and diagnosis. If a patient's medical record includes medical parameters, the electronic device may determine which diagnosis the medical parameters are most associated with based on the similarity coefficient between the medical parameters and various types of diagnosis. The electronic device may provide the user with the recommended diagnosis list based on the determination result, so that the user may select the diagnosis that needs to be executed. If the recommended diagnosis has a corresponding additional diagnosis, the electronic device may also add the additional diagnosis to the recommended diagnosis list for the user's reference. The recommended diagnosis list may also include the cost details calculated based on the insurance data, so that the user may select an appropriate diagnosis based on financial ability. In addition, the electronic device may also access the external server to automatically register the data that the hospital has not registered in the medical record for medical staff or the patient to confirm. Since the electronic device may automatically obtain the data used to generate the recommended diagnosis list, the electronic device may significantly reduce the burden of medical staff registering the data and improve the accuracy of accounting and clinical records.

1. An electronic device for providing a recommended diagnosis, comprising:
a transceiver;
a storage medium, storing a first similarity between a first diagnosis and a first medical parameter and a second similarity between the first diagnosis and a second medical parameter; and
a processor, coupled to the storage medium and the transceiver, wherein the processor is configured to execute:
receiving a medical record of a patient through the transceiver;
in response to the medical record comprising the first medical parameter and the second medical parameter, adding the first similarity to the second similarity to generate a first similarity coefficient and generating a recommended diagnosis list corresponding to the medical record based on the first diagnosis selected by the first similarity coefficient, wherein the recommended diagnosis list comprises the first diagnosis corresponding to the first similarity; and
outputting the recommended diagnosis list through the transceiver.

2-4. (canceled)

5. The electronic device according to claim 1, wherein the processor is further configured to execute:
in response to the first similarity coefficient corresponding to the first diagnosis being greater than a second similarity coefficient corresponding to a second diagnosis, selecting the first diagnosis from the first diagnosis and the second diagnosis as a selected diagnosis corresponding to the first medical parameter; and
generating the recommended diagnosis list based on the selected diagnosis.

6. The electronic device according to claim 1, wherein the medical record comprises a plurality of medical parameters, wherein the processor is further configured to execute:
calculating a number of selected diagnosis based on a weight corresponding to the first medical parameter;
selecting the first medical parameter from the plurality of medical parameters based on the number of selected diagnosis to obtain a selected diagnosis corresponding to the first medical parameter; and
generating the recommended diagnosis list based on the selected diagnosis.

7. The electronic device according to claim 6, wherein the processor determines the weight corresponding to the first medical parameter based on a department corresponding to the medical record.

8. The electronic device according to claim 1, wherein the storage medium further stores a list corresponding to the first diagnosis, wherein the processor is further configured to execute:
obtaining an additional diagnosis corresponding to the first diagnosis based on the list; and
adding the additional diagnosis to the recommended diagnosis list.

9. The electronic device according to claim 1, wherein the storage medium further stores a blacklist corresponding to the first diagnosis, wherein the processor is further configured to execute:
removing a recommended diagnosis in the recommended diagnosis list based on the blacklist.

10. The electronic device according to claim 1, wherein the processor accesses an external server through the transceiver to add updated data to the medical record.

11. The electronic device according to claim 10, wherein the processor outputs a report comprising the updated data through the transceiver based on a default text format.

12. The electronic device according to claim 1, wherein the first medical parameter corresponds to one of the following variables: patient features, inspections, medications, and treatment or surgery.

13. The electronic device according to claim 1, wherein the storage medium further stores insurance data, wherein the processor calculates a cost of the first diagnosis based on the insurance data, and outputs a report comprising the cost through the transceiver.

14. A method for providing a recommended diagnosis, comprising:
obtaining a first similarity between a first diagnosis and a first medical parameter and a second similarity between the first diagnosis and a second medical parameter;
obtaining a medical record of a patient;
in response to the medical record comprising the first medical parameter and the second medical parameter, adding the first similarity to the second similarity to generate a first similarity coefficient and generating a recommended diagnosis list corresponding to the medical record based on the first diagnosis selected by the first similarity coefficient, wherein the recommended diagnosis list comprises the first diagnosis corresponding to the first similarity; and
outputting the recommended diagnosis list.
